(19) United States
(12) Patent Application Publication
(10) Pub. No.: US 2005/0106178 A1
(43) Pub. Date: May 19, 2005

(54) Adjuvanted influenza vaccine

(75) Inventor: Derek O'Hagan, Berkeley, CA (US)

Correspondence Address: Chiron Corporation;Intellectual Property - R440, P.O. Box 8097, Emeryville, CA, 94662-8097, US

(73) Assignee: Chiron Corporation

(21) Appl. No.: 10/769,628

(22) Filed: Jan. 29, 2004

Related U.S. Application Data
(60) Provisional application No. 60/443,985, filed on Jan. 30, 2003.

(51) Int. Cl. A61K 39/145; A61K 39/02

(52) U.S. Cl. 424/209.1; 424/235.1

(57) ABSTRACT

The invention relates to compositions comprising one or more influenza antigens, one or more detoxified ADP-ribosylating proteins, and one or more chitosans. The invention also relates to methods of using these compositions to generate an immune response to influenza.

### CROSS-REFERENCE TO RELATED APPLICATIONS

This application claims the benefit of U.S. Ser. No. 60/443,985 filed Jan. 30, 2003, which application is hereby incorporated by reference in its entirety.

### TECHNICAL FIELD

This invention is in the field of vaccines, particularly against influenza infection and disease.

### BACKGROUND

Inactivated influenza vaccines are widely available. See, e.g. see Chapter 21 of Vaccines, eds. Plotkin & Orenstein, 3^{rd} edition (1999) ISBN 0-7216-7443-7. Currently available inactivated influenza vaccines are typically administered intramuscularly, although immunogenicity has been observed when administered by the subcutaneous, intradermal, respiratory tract and oral routes.

An attractive route of administration for various flu vaccines is the intranasal route, and this has received much attention. See, e.g., Gluck et al. (2002) J. Aerosol Med 15:221-228; U.S. Pat. No. 6,534,065; Harper et al. (2003) MMWR Recomm Rep. Sep. 26, 2003;52(RR-13):1-8.

It is an object of the invention to provide modified and improved flu vaccines, and in particular to provide flu vaccines suitable for intranasal or other mucosal administration.

### SUMMARY

The present invention is based on the surprising and unexpected discovery that the use of chitosan in combination with a ribosylating toxin serves to enhance the immunogenicity of influenza antigen(s). The use of such combinations provides a safe and effective approach for enhancing the immunogenicity of a wide variety of antigens.

In one aspect, the invention includes a composition comprising an influenza antigen; a detoxified ADP-ribosylating protein; and a chitosan. In certain embodiments, the influenza antigen comprises one or more surface antigens (e.g., haemagglutinin (HA) and/or neuraminidase. In any of the compositions described herein, the detoxified ADP-ribosylating protein may be a detoxified diphtheria toxin protein, a detoxified exotoxin A protein, a detoxified cholera toxin (CT) protein; a detoxified heat-labile enterotoxin (LT) toxin protein; and/or a detoxified pertussis toxin (PT) protein, including for example, LTK7, LTX53, LTK63, LTY63, LTR72, LTX97, LTX104,LTS106, LTK112, LTG192, CTK7, CTK11, CTX53, CTF61, CTK63, CTY63, CTX97, CTX104, CTK112, CTS106, PTK9, PTG129 and combinations thereof. Furthermore, in any of the compositions described herein, the chitosan may be at least 75% deacteylated and/or alkylated (e.g., trialkylated). Any of the compositions described herein may further comprise one or more additional antigens and/or one or more additional adjvuants.

In another aspect, one or more of the components of the compositions described herein are provided as polynucleotides encoding that component, for example, a polynucleotide encoding an influenza antigen.

The compositions described herein may be adapted for mucosal administration, for example intranasal administration via nasal spray or nasal drops.

In yet another aspect, the invention includes any of the compositions described herein in combination with a dispensing device, for example a dispensing device that is adapted to deliver the composition intranasally.

In yet another aspect, a kit comprising any of the compositions described herein is provided. In certain embodiments, the components of the kit are supplied separately and, when combined or reconstituted, the composition is suitable for mucosal administration.

In still another aspect, the invention includes a method of generating an immune response to influenza in a subject, the method comprising: administering to the subject any of the compositions described herein. In certain embodiments, the immune response protects the subject against influenza infection or disease.

These and other embodiments of the present invention will readily occur to those of ordinary skill in the art in view of the disclosure herein.

### BRIEF DESCRIPTION OF THE DRAWINGS

FIG. 1 depicts the structure of a chitosan made up of glucosamine monomers.

FIG. 2 depicts the structure of a chitin, from which chitosan can be derived.

FIG. 3 depicts the structure of a trimethylchitosan monomer.

FIG. 4 is a graph depicting IgG titers after immunization with haemagglutinin (HA)-containing compositions as described herein.

FIG. 5 is a graph depicting Haemagglutination inhibition (HI) titers from the same animals as shown in FIG. 4.

### DETAILED DESCRIPTION

Described herein are immunogenic compositions comprising: (a) one or more influenza antigens, (b) one or more detoxified ADP-ribosylating toxins, and (c) a chitosan. The inclusion of chitosan allows a lower dose of the toxin to be used, thereby improving safety. The compositions are preferably suitable for mucosal administration, e.g. intranasal administration.

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B. N. Raven Press, New York, N.Y.

As used in this specification and the appended claims, the singular forms “a,” “an” and “the” include plural references unless the content clearly dictates otherwise. Thus, for example, reference to “an antigen” includes a mixture of two or more such antigens.

The disclosures of all patents, patent applications and publications mentioned herein are hereby incorporated by reference in their entireties.

A. Definitions

Prior to setting forth the invention certain terms that will be used hereinafter are discussed below.

The terms “Polypeptide” and “protein” refer to a polymer of amino acid residues and are not limited to a minimum length of the product. Thus, peptides, oligopeptides, dimers, multimers, and the like, are included within these terms. Both full-length proteins and fragments thereof are encompassed by these terms. The terms also include postexpression modifications of the polypeptide, for example, glycosylation, acetylation, phosphorylation and the like. Furthermore, for purposes of the present invention, a “polypeptide” refers to a protein that includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts that produce the proteins or errors due to PCR amplification. Furthermore, modifications may be made that have one or more of the following effects: reducing toxicity; facilitating cell processing (e.g., secretion, antigen presentation, etc.); and facilitating presentation to B-cells and/or T-cells.

A “fusion molecule” refers to a molecule in which two or more subunit molecules are linked, preferably covalently. The subunit molecules can be the same chemical type of molecule, or can be different chemical types of molecules. Examples of the fusion molecules include, but are not limited to, fusion polypeptides (for example, a fusion between two or more antigens). See, also, Sambrook et al., supra and Ausubel et al., supra for methods of making fusion molecules.

An “antigen” refers to a molecule containing one or more epitopes (either linear, conformational or both) that will stimulate a host's immune system to make a humoral and/or cellular antigen-specific response. The term is used interchangeably with the term “immunogen.” Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term “antigen” denotes both subunit antigens, (i.e., antigens which are separate and discrete from a whole organism with which the antigen is associated in nature), as well as, killed, attenuated or inactivated bacteria, viruses, fungi, parasites or other microbes as well as tumor antigens, including extracellular domains of cell surface receptors and intracellular portions that may contain T-cell epitopes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses an antigen or antigenic determinant in vivo, such as in gene therapy and DNA immunization applications, is also included in the definition of antigen herein.

Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Nat'l Acad Sci. USA 81:3998-4002; Geysen et al. (1986) Molec. Immunol 23:709-715.

Similarly, conformational epitopes are readily identified by determining spatial conformation of amino acids such as by, e.g., x-ray crystallography and nuclear magnetic resonance. See, e.g., Epitope Mapping Protocols, supra.

Furthermore, for purposes of the present invention, an “antigen” refers to a protein that includes modifications, such as deletions, additions and substitutions (generally conservative in nature), to the native sequence, so long as the protein maintains the ability to elicit an immunological response. These modifications may be deliberate, as through site-directed mutagenesis, or may be accidental, such as through mutations of hosts that produce the antigens.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+T-cells. In addition, a chemokine response may be induced by various white blood or endothelial cells in response to an administered antigen.

A composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376. Recent methods of measuring cell-mediated immune response include measurement of intracellular cytokines or cytokine secretion by T-cell populations (e.g., by ELISPOT technique), or by measurement of epitope specific T-cells (e.g., by the tetramer technique)(reviewed by McMichael, A. J., and O'Callaghan, C. A., J. Exp. Med. 187(9):1367-1371, 1998; Mcheyzer-Williams, M. G., et al, Immunol. Rev. 150:5-21, 1996; Lalvani, A., et al, J. Exp. Med. 186:859-865, 1997).

Thus, an immunological response as used herein may be one that stimulates CTLs, and/or the production or activation of helper T-cells. The production of chemokines and/or cytokines may also be stimulated. The antigen of interest may also elicit an antibody-mediated immune response. Hence, an immunological response may include one or more of the following effects: the production of antibodies (e.g., IgA or IgG) by B-cells ; and/or the activation of suppressor, cytotoxic, or helper T-cells and/or T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

An “immunogenic composition” refers to a composition that comprises an antigenic molecule where administration of the composition to a subject results in the development in the subject of a humoral and/or a cellular immune response to the antigenic molecule of interest. The immunogenic composition can be introduced directly into a recipient subject, such as by injection, inhalation, oral, intranasal or any other parenteral or mucosal route of administration.

“Subunit vaccine” refers to a vaccine composition that includes one or more selected antigens but not all antigens, derived from or homologous to, an antigen from a pathogen of interest such as from a virus, bacterium, parasite or fungus. Such a composition is substantially free of intact pathogen cells or pathogenic particles, or the lysate of such cells or particles. Thus, a “subunit vaccine” can be prepared from at least partially purified (preferably substantially purified) immunogenic polypeptides from the pathogen, or analogs thereof. The method of obtaining an antigen included in the subunit vaccine can thus include standard purification techniques, recombinant production, or synthetic production.

“Parenteral administration” refers to introduction into the body outside the digestive tract, such as by subcutaneous, intramuscular, intradermal or intravenous administration. This is to be contrasted with delivery to a mucosal surface, such as oral, nasal, vaginal or rectal. Thus, “mucosal administration” refers to introduction into the body via any mucosal surface, such as intragastrically, pulmonarily, transdermally, intestinally, ocularly, intranasally, orally, vaginally, rectally or the like.

“Co-administration” refers to introduction into a body or target cell of two or more compositions. The term includes administration in any order or concurrently.

An “immuno-modulatory factor” refers to a molecule, for example a protein that is capable of modulating (particularly enhancing) an immune response. Non-limiting examples of immunomodulatory factors include lymphokines (also known as cytokines), such as IL-6, TGF-□, IL-1, IL-2, IL-3, etc.); and chemokines (e.g., secreted proteins such as macrophage inhibiting factor). Certain cytokines, for example TRANCE, flt-3L, and a secreted form of CD40L are capable of enhancing the immunostimulatory capacity of APCs. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1α), interleukin-11 (IL-11), MIP-1α, leukemia inhibitory factor (LIF), c-kit ligand, thrombopoietin (TPO), CD40 ligand (CD40L), tumor necrosis factor-related activation-induced cytokine (TRANCE) and flt3 ligand (flt-3L). Cytokines are commercially available from several vendors such as, for example, Genzyme (Framingham, Mass.), Amgen (Thousand Oaks, Calif.), R&D Systems and Immunex (Seattle, Wash.). The sequences of many of these molecules are also available, for example, from the GenBank database. It is intended, although not always explicitly stated, that molecules having similar biological activity as wild-type or purified cytokines (e.g., recombinantly produced or mutants thereof) and nucleic acid encoding these molecules are intended to be used within the spirit and scope of the invention. Immunomodulatory factors can be included with one, some or all of the compositions described herein or can be employed as separate formulations.

“Subject” refers to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

“Vertebrate subject” refers to any member of the subphylum cordata, including, without limitation, mammals such as cattle, sheep, pigs, goats, horses, and humans; domestic animals such as dogs and cats; and birds, including domestic, wild and game birds such as cocks and hens including chickens, turkeys and other gallinaceous birds. The term does not denote a particular age. Thus, both adult and newborn animals are intended to be covered.

“Pharmaceutically acceptable” or “pharmacologically acceptable” refers to a material which is not biologically or otherwise undesirable, i.e., the material may be administered to an individual in a formulation or composition without causing any undesirable biological effects or interacting in a deleterious manner with any of the components of the composition in which it is contained.

As used herein, “treatment” refers to any of (i) the prevention of infection or reinfection, as in a traditional vaccine, (ii) the reduction or elimination of symptoms, and (iii) the substantial or complete elimination of the pathogen or disorder in question. Treatment may be effected prophylactically (prior to infection) or therapeutically (following infection).

The term “comprising” means “including” as well as “consisting” e.g. a composition “comprising” X may consist exclusively of X or may include something additional e.g. X+Y.

The term “about,” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

B. Antigens

B.1. Influenza Antigens(s)

Any influenza antigen(s) can be used in the compositions and methods described herein. For example, influenza antigens may be surface antigens such as haemagglutinin (HA) and/or neuraminidase (NA) antigens. The antigens can form part of a whole influenza vaccine composition, or they can be present as purified or substantially purified antigens. Purified antigens (e.g., haemagglutinin and neuraminidase antigens) may be present in the form of rosettes, for example rosette particles with a radius in the range 10 to 25 nanometers.

Influenza antigen(s) may be obtained from a single viral strain, or from a plurality of strains. Thus, the compositions described herein may include any number of antigens from any number of different strains. For example, multiple proteins representing a specific antigen may be included, where each protein is derived from a different strain. Alternatively, multiple proteins representing various antigens may be included, where each protein is derived from one or from various different strains.

For vaccine production, influenza virus has traditionally been grown in embryonated hens' eggs and purified by zonal centrifugation or chromatography. The virus can be included in vaccines as whole virions, but it is more common to disrupt the virus in order to decrease toxicity and reactogenicity. Treatment with detergent or organic solvent, for instance, yields “split” vaccines in which immunogenic surface glycoproteins are retained. Further purification gives “subunit” or purified surface antigen vaccines, which consist mainly of haemagglutinin (HA) and neuraminidase (NA).

Because of problems associated with retention of allergenic proteins, more modern production techniques have moved away from the use of eggs and towards cell culture, as recommended by the WHO in 1995. Typical cell lines for influenza culture include MDCK and Vero cell lines. To reduce contamination, cells are preferably grown in a serum-free or protein-free medium. To reduce contamination even further, and to reduce host cell DNA levels, virions can be treated by a process involving treatment with DNAse and cationic detergent. See, e.g., U.S. Pat. No. 5,948,410.

Thus, influenza antigens can be produced recombinantly using standard techniques. Selected coding sequences can be cloned into any suitable expression vector for expression. The expressed product can optionally be purified prior to mucosal administration. Briefly, a polynucleotide encoding these proteins can be introduced into an expression vector that can be expressed in a suitable expression system. A variety of bacterial, yeast, mammalian, insect and plant expression systems are available in the art and any such expression system can be used. Optionally, a polynucleotide encoding these antigens can be translated in a cell-free translation system. Such methods are well known in the art. The proteins also can be constructed by solid phase protein synthesis. If desired, the polypeptides also can contain other amino acid sequences, such as amino acid linkers or signal sequences, as well as ligands useful in protein purification, such as glutathione-S-transferase and staphylococcal protein A. Alternatively, antigens of interest can be purchased from commercial sources.

Other modern approaches to influenza vaccination are reviewed in, for example, Palese et al. (2002) J. Clin Invest 110:9-13.

Any of these influenza antigens prepared in any of these ways may be used with the invention, but it is preferred (a) to use purified HA and, optionally, purified NA, and (b) to use antigens purified from cell lines rather than from eggs.

Whatever antigen(s) is/are used, it is preferred that it/they is/are selected to offer suitable coverage of existing strains. Updated guidelines on strains and subtypes are regularly issued by bodies such as the WHO, but in general it is preferred to include antigens from more than one strains, and containing a least one type A virus (e.g. A/H1N1 and A/H3N2) and at least one type B virus.

In one embodiment, the antigens are selected from a flu strain that is capable of or has the potential for causing a pandemic outbreak. Typically, a pandemic flu strain contains a haemagglutinin protein which is different from currently circulating strains or which has not been evident in the human population for an extended period of time. Examples of haemagglutinin proteins potentially associated with a pandemic flu strain include H2, H5, H6 or H9. Such antigens are discussed, for instance, in Hilleman, “Realities and enigmas of human viral influenza: pathogenesis, epidemiology and control”, Vaccine (2002) 20:3068-3087 and Ha, et al., “X-ray structures of H5 avian and H9 swine influenza virus haemagglutinins bound to avian and human receptor analogs”, PNAS (2001) 98(20): 11181-11186.

As an alternative to using glycoprotein antigens in the compositions described herein, nucleic acids encoding the antigen(s) may be used instead. See, e.g., Palese et al, supra; Ulmer et al. (2002) Vaccine 20 Suppl 2:S74-76. One or more of the antigen components of the mixture may be replaced by nucleic acid (preferably DNA, e.g. in the form of a plasmid) that encodes the protein. Combinations of nucleic acid and polypeptide antigens may also be used.

B.2. Additional Antigens

The compositions of the invention typically include at least one influenza antigen. However, the invention can also be applied to other antigens, in addition to influenza antigens. The composition of the invention may thus include one or more of the following antigens, in place of or in addition to the influenza antigen(s) described above:
antigens from Helicobacter pylori such as CagA (see, e.g., Covacci & Rappuoli (2000) J Exp. Med. 19:5 87-592; W093/18150; Covacci et al. (1993) Proc. Natl. Acad. Sci. USA 90: 5791-5795; Tummuru et al. (1994) Infect. Immun. 61:1799-1809); VacA (see, e.g., Marchetti et al. (1998) Vaccine 16:33-37; Telford et al (1994) J Exp. Med. 179:1653-1658); NAP (see, e.g., Evans et al (1995) Gene 153:123-127; International Publications WO 96/01272; WO 96/01273 (esp. SEQ ID NO:6); W097/25429), HopX (see, e.g., International Publication WO 98/04702), HopY (see, e.g., International Publication WO 98/04702) and/or urease.
a saccharide antigen from Streptococcus pneumoniae (see, e.g., Watson (2000) Pediatr. Infect. Dis J 19:331-332; Rubin (2000) Pediatr Clin North Am 47:269-285; Jedrzejas (2001) MicrohiolMolBiolRev 65:187-207).
an antigen from hepatitis A virus, such as inactivated virus (see, e.g., Bell (2000) Pediatr Infect Dis J 19:1187-1188; Iwarson (1995) APMIS 103:321-326)
an antigen from hepatitis B virus, such as the surface and/or core antigens (see, e.g., Gerlich et al. (1990) Vaccine 8 Suppl:S63-68 & 79-80), with surface antigen preferably being adsorbed onto an aluminum phosphate (see, e.g., International Publication W093/24148).
a protein from serogroup B of N. meningitidis (see, e.g., International Publications W0 99/24578, W0 99/36544; W099/57280; WO 00/22430; Tettelin et al. (2000) Science 287:1809-1815; Pizza et al (2000) Science 287:1816-1820).
an OMV preparation from serogroup B of N.meningitidis (see, e.g., International Publication WO 01/52885; Bjune et al. (1991) Lancet 338(8775):1093-1096; Fukasawa et al. (1999) Vaccine 17:2951-2958; Rosenqvist et aL (1998) Dev. Biol. Stand. 92:323-333).
a saccharide antigen from Hemophilus influenzae B, preferably non-adsorbed or adsorbed onto an aluminum phosphate (see, e.g., International Publication W0 97/00697).
an antigen from hepatitis C virus (see, e.g., Hsu et al. (1999) Clin Liver Dis 3:901-915).
an antigen from N. gonorrhoeae (see, e.g., International Publications W0 99/24578, W0 99/36544; W099/57280; WO 00/22430).
an antigen from Chlamydia pneumoniae (see, e.g., International Publications W0 02/02606; WO 00/27994; W0 99/27105; WO 00/37494; Kalman et al. (1999) Nature Genetics 21:385-389; Read et al. (2000) Nucleic Acids Res 28:1397-406; Shirai et al. (2000) J Infect. Dis. 181 (Suppl 3):S524-S527).
an antigen from Chlamydia trachomatis (see, e.g., W0 99/28475).
an antigen from Porphyromonas gingivalis (see, e.g., Ross et al. (2001) Vaccine 19:41354142).
polio antigen(s) (see, e.g., Sutter et al. (2000) Pediatr Clin North Am 47:287-308; Zimmerman & Spann (1999) Am Fam Physician 59:113-118,125-126) such as IPV.
rabies antigen(s) (see, e.g., Dreesen (1997) Vaccine 15 Suppl:S2-6) such as lyophilised inactivated virus, RabAvert™ as described in MMWR Morb Mortal Wkly Rep Jan. 16, 1998;47(1):12,19].
measles, mumps and/or rubella antigens (see, e.g. Chapters 12, 13 & 17 of Vaccines, eds. Plotkin & Orenstein, 3^{rd} edition (1999) ISBN 0-7216-7443-7)
an antigen from Moraxella catarrhalis (see, e.g., McMichael (2000) Vaccine 19 Suppl 1: S101-107).
an antigen from Streptococcus agalactiae (group B streptococcus) (see, e.g., Schuchat (1999) Lancet 353(9146):51-6; International Publication W002/34771).
an antigen from Streptococcus pyogenes (group A streptococcus) (see, e.g., International Publication W0 02/34771; Dale (1999) Infect Dis Clin North Am 13:227-43, viii; Ferretti et al. (2001) PNAS USA 98: 4658-4663).
an antigen from Staphylococcus aureus (see, e.g., Kuroda et al (2001) Lancet 357(9264):1225-1240; see also pages 1218-1219).
antigen(s) from a paramyxovirus such as respiratory syncytial virus (RSV, see, e.g., Anderson (2000) Vaccine 19 Suppl I :S59-65; Kahn (2000) Curr Opin Pediatr 12:257-262) and/or parainfluenza virus (PIV3, see, e.g,. Crowe (1995) Vaccine 13:415-421).
an antigen from Bacillus anthracis (see, e.g., J Toxicol Clin Toxicol (2001) 39:85-100; Demicheli et al. (1998) Vaccine 16:880-884; Stepanov et al. (1996) J Biotechnol 44:155-160).
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B19.
a tetanus toxoid (see, e.g. chapter 18 of Vaccines, eds. Plotkin & Orenstein, 3^{rd} edition (1999) ISBN 0-7216-7443-7)
pertussis holotoxin (PT) and filamentous hemagglutinin (FHA) from B.pertussis, optionally also in combination with pertactin and/or agglutinogens 2 and 3 (see, e.g., Gustafasson et al. (1996) N. Engl. J Med. 334:349-355; Rappuoli et al (1991) TIBTECH 9:232-238).
cellular pertussis antigen.

The mixture may comprise one or more of these further antigens, which may be detoxified where necessary (e.g. detoxification of pertussis toxin by chemical and/or genetic means).

Where a diphtheria antigen is included in the mixture it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the mixture will typically be present at a concentration of at least 1 μg/ml each. The concentration of antigen per dose is typically between about 1 μg/dose and about 20 μg/dose, for example, 5 μg/dose, 7 μg/dose, 10 μg/dose or 15 μg/dose. In general, the concentration of any given antigen will be sufficient to elicit an immune response against that antigen.

As an alternative to using proteins antigens in the mixture, nucleic acid encoding the antigen may be used. Protein components of the mixture may thus be replaced by nucleic acid (preferably DNA e.g. in the form of a plasmid) that encodes the protein. Similarly, compositions of the invention may comprise proteins which mimic saccharide antigens e.g. mimotopes (see, e.g., Charalambous & Feavers (2001) J Med Microbiol 50:937-939) or anti-idiotype antibodies. These may replace individual saccharine components, or may supplement them.

C. Adjuvants

C.1. Detoxified ADP-Ribosylating Toxins

ADP-ribosylating bacterial exotoxins that catalyze the transfer of an ADP-ribose unit from NAD⁺ to a target protein are widely known. Examples include diphtheria toxin (Corynebacterium diphtheriae), exotoxin A (Pseudomonas aeruginosa), cholera toxin (CT; Vibrio cholerae), heat-labile enterotoxin (LT; E. coli) and pertussis toxin (PT). Further examples are disclosed in WO 02/079242 and The Comprehensive Sourcebook of Bacterial Protein Toxins (Alouf & Freer) ISBN 0120530759.

The toxins are typically divided into two functionally distinct domains—A and B. The A subunit is responsible for the toxic enzymatic activity, whereas the B subunit is responsible for cellular binding. The subunits might be domains on the same polypeptide chain, or might be separate polypeptide chains. The subunits may themselves be oligomers e.g. the A subunit of CT consists of A₁ and A₂ which are linked by a disulphide bond, and its B subunit is a homopentamer. Typically, initial contact with a target cell is mediated by the B subunit and then subunit A alone enters the cell.

The toxins are typically immunogenic, but their inclusion in vaccines is hampered by their toxicity. To remove toxicity without also removing immunogenicity, the toxins have been treated with chemicals such as glutaraldehyde or formaldehyde. A more rational approach relies on site-directed mutagenesis of key active site residues to remove toxic enzymatic activity whilst retaining immunogenicity (see, e.g. International Publication WO 93/1302 (CT and LT); European Patent Applications 0306618; 0322533; and 0322115 (PT), Del Giudice et al. (1999) Vaccine 17 Suppl. 2:S44-52). Current acellular whooping cough vaccines include a form of pertussis toxin with two amino acid substitutions (Arg⁹→Lys and Glu¹²⁹ →Gly; ‘PT-9K/129G’). See, e.g., European Patent Application 0396964.

As well as their immunogenic properties, the toxins have been used as adjuvants. Parenteral adjuvanticity was first observed in 1972 (Northrup & Fauci (1972) J. Infect. Dis. 125:672ff) and mucosal adjuvanticity in 1984 (Elson & Ealding (1984) J. Immunol. 133:2892ff and 132:2736ff). It was surprisingly found in 1993 that the detoxified forms of the toxins retain adjuvanticity (International Publication WO 95/17211).

The compositions of the invention include a detoxified ADP-ribosylating toxin. The toxin may be diphtheria toxin, Pseudomonas exotoxin A or pertussis toxin, but is preferably cholera toxin (CT) or, more preferably, E.coli heat-labile enterotoxin (LT). Other toxins that can be used are those disclosed in WO 02/079242 (SEQ IDs 1 to 7 therein, and mutants thereof).

Detoxification of these toxins without loss of immunogenic and/or adjuvant activity can be achieved by any suitable means, with mutagenesis being preferred. Mutagenesis may involve one or more substitutions, deletions and/or insertions.

Preferred detoxified mutants include one or more amino acid substitutions as compared to a wild type protein. Pursuant to accepted nomenclature, the detoxified mutants are referred to as by the protein abbreviation (e.g., LT, CT, PT, etc.), followed by a particular single letter amino acid or an “X” to represent any amino acid substitution, followed by the amino acid residue as numbered relative to wild-type, for example as disclosed in Domenighini et al., Molecular Microbiology (1995) 15(6): 1165-1167. Multiple amino acid substitutions will follow the same accepted nomenclature, e.g., LTK7K63.

Non-limiting examples of detoxified mutants include LT having a mutation at residue Arg-7 (e.g. a Lys substitution, also referred to as LTK7); CT having a mutation at residue Arg-7 (e.g. a Lys substitution, also referred to as CTK7); CT having a mutation at residue Arg-11 (e.g. a Lys substitution, also referred to as CTK11); LT having a mutation at Val-53 (also referred to as LTX53); CT having a mutation at Val-53 (also referred to as CTX53); CT having a mutation at residue Ser-61 (e.g. a Phe substitution, also referred to as CTF61); LT having a mutation at residue Ser-63 (e.g. a Lys or Tyr substitution, also referred to as LTK63 and LTY63, where K63 described in Chapter 5 of Del Giudice et al. (1998) Molecular Aspects of Medicine, vol. 19, number 1; Y63 described in Park et al (2000) Exp. Mol. Med. 32:72-78); CT having a mutation at residue Ser-63 (e.g. a Lys or Tyr substitution, also referred to as CTK63 and CTY63); LT having a mutation at residue Ala-72 (e.g. an Arg substitution,R72, described in International Publication WO 98/18928); LT having a mutation at Val-97 (also referred to as LTX97); CT having a mutation at Val-97 (also referred to as CTX97); LT having a mutation at Tyr-104 (also referred to as LTX104); CT having a mutation at Tyr-104 (also referred to as CTX104); LT having a mutation at residue Pro-106 (e.g. a Ser substitution, also referred to as LTS106); CT having a mutation at residue Pro-106 (e.g. a Ser substitution, also referred to as CTS106); LT having a mutation at Glu-112 (e.g. a Lys substitution, also referred to as LTK112); CT having a mutation at Glu-112 (e.g. a Lys substitution, also referred to as CTK112); LT having a mutation at residue Arg-192 (e.g. a Gly substitution, also referred to as LTG192); PT having a mutation at residue Arg-9 (e.g. a Lys substitution, also referred to as PTK9); PT having a mutation at Glu-129 (e.g. a Gly substitution, also referred to as PTG129); combinations thereof and any of the mutants disclosed in International Publication WO 93/13202.

As noted above, the amino acid sequences for CT and LT are described in Domenighini et al., Molecular Microbiology (1995) 15(6): 1165-1167. This reference and the CT and LT sequence alignment disclosed therein are incorporated herein in their entirety.

These mutations may be combined e.g. Arg-9-Lys+Glu-129-Gly in PT, or LT with both a D53 and a K63 mutation, etc.

LT with a mutation at residue 63 and/or 72 is a preferred detoxified toxin. The LTK63 and LTR72 toxins are particularly preferred. See, e.g., Pizza et al. (2000) Int. J. Med. Microbiol. 290:455-461.

It will be appreciated that the numbering of these residues is based on prototype sequences and that, for example, although Ser-63 may not actually be the 63rd amino acid in a given LT variant, an alignment of amino acid sequences will reveal the location corresponding to Ser-63.

The detoxified toxins may be in the form of A and/or B subunits as appropriate for activity.

As with the antigens described above, the ADP-ribosylating proteins of the compositions described herein may be administered as polypeptides or as nucleic acids encoding these proteins. Therefore, one or more of the ADP-ribosylating protein components of the mixture may be replaced by nucleic acid (preferably DNA, e.g. in the form of a plasmid) that encodes the protein. Combinations of nucleic acid and polypeptide antigens may also be used.

C.2. Chitosan

In addition to one or more influenza antigens and one or more ribosylating toxin adjuvants, the compositions described herein also comprise one or more chitosans.

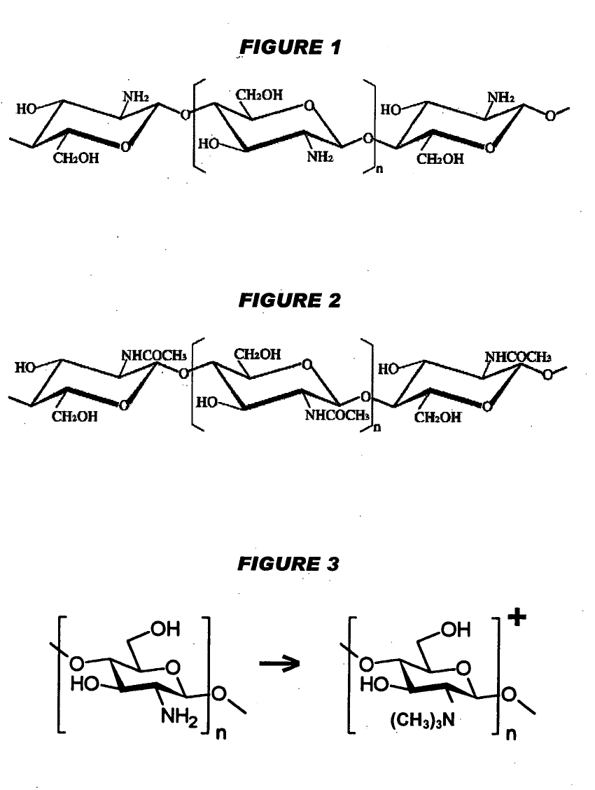

Chitosan (also known as mycosin) is typically a polymer of incompletely N-acetylated (1-4)-β-linked D-glucosamine residues (FIG. 1) and is usually derived from natural sources of chitin, such as exoskeletons of crustaceans. An exemplary exoskeletal polymer chitin is shown in FIG. 2. Chitosan is typically not fully deacetylated and, as such, unlike chitin, is soluble in dilute aqueous acetic and formic acids.

Chitosan has found wide applicability in non-vaccine pharmaceutical fields. See, e.g., Singla & Chawla (2001) J. Pharm. Pharmacol. 53:1047-1067. In addition, the use of chitosan as an adjuvant has been reported. See, e.g., U.S. Pat. Nos. 6,534,065; 6,912,000; 6,048,536; 6,136,606; 6,391,318; International Publication Nos. WO 96/09805, WO 96/10421, WO 97/01330; WO 97/16208, WO 97/20576; WO 98/42374; WO 99/27960; WO 01/35994; van der Lubben et al. (2001) Eur. J. Pharm Sci. 14:201-207; Le Buanec et al. (2001) Biomed. Pharmacother. 55:316-320; Seferian & Martinez et al. (2000) Vaccine 19:661-668; Jabbal-Gill et al. (1998) Vaccine 19:2039-2046; and Marcinkiewicz et al. (1991) Arch. Immunol. Ther. Exp. (Warsz) 39:127-132.

A variety of chitosan polymers and derivatives thereof will find use in the present invention. Non-limiting examples of chitosans that may be used include, oligosaccharides with relatively low molecular weights ranging from around 5,000-100,000 to polymers of relatively high molecular weight (e.g. 600,000-1,000,000). Preferably the weight average molecular weight of the chitosan (or chitosan) salt is in the range 11,000 to 49,000, more preferably 15,000 to 35,000, particularly 17,000 to 32,000, and most particularly 20,000 to 32,000.

Generally, the chitosan and derivatives thereof used with the invention are preferably at least 75% deacetylated, for example 75%-100% (or any value therebetween), more preferably 75-90% deacetylated, particular examples being 83%, 84%, 85%, 86% and 87% deacetylation.

Derivates and salts of chitosans also find use in the present invention. As shown in FIG. 1, the repeating glucosamine monomer of chitosan contains an amine group. This group may exist as free amine (—NH₂) or as cationic amine (—NH₃⁺), with protonation affecting the polymer's solubility. The amine groups are chemically active and one or more amine groups can be substituted. Of particular interest for the invention, the amine groups can be substituted with one or more alkyl group (‘A’ e.g. methyl, ethyl, propyl, butyl, pentyl, etc.) e.g. —NHA, —NH₂A⁺, —NA₂A², —NHA¹A²⁺, —NA¹A²A³⁺. Preferred derivatives are tri-alkylated and particularly preferred derivatives are trimethylated (i.e. trimethylchitosan, or ‘TMC’—FIG. 3). These derivatives much higher aqueous solubility than unmodified chitosan over a broader pH range.

It is to be understood that not every amine in a chitosan polymer needs to be substituted in this way. The degree of substitution along the length of the chitosan chain can be determined by ¹H-NMR and can be controlled by means of the number and duration of reaction steps. See, e.g., Hwang et al. (2002) J. Agric. Food Chem. 50:1876-1882. Because the monomers are typically not all deacetylated and because substitution reactions are not usually 100% efficient, it is rare that every monomer in the chitosan polymer will be substituted with an alkylated amine. Preferably, at least 10% (e.g. at least 20%, 30%, 40%, 50%, 60%, 70%, 80%, 90%, 95% or more) of glucosamine monomers have a substituted amine. Thus, alkylated chitosan derivatives used in the invention may therefore have amide and/or non-alkylated groups on some monomer units, and chitosan may possess some amide groups.

Where a cationic chitosan or derivative is used, it will be in the form of a salt e.g. chloride or lactate. Generally, the acid addition salt is formed by reaction with a suitable pharmaceutically acceptable acid, for instance a mineral acid or an organic acid, such as a carboxylic or dicarboxylic acid, or a dicarboxy-amino acid. Non-limiting examples of acid addition salts are those formed with acids such as hydrochloric, nitric, sulphuric, acetic, phosphoric, toluenesulphonic, methanesulphonic, benzenesulphonic, lactic, malic, maleic, succinic, lactobionic, fiunaric and isethionic acids, glutamic acid and aspartic acid. Furthermore, the acid is preferably compatible with the antigen and does not have a significant adverse effect on the adjuvant properties of the chitosan. Preferred acid addition salts are carboxylate salts such as glutamate salts.

The chitosan or derivative can take various physical forms, for example, in solution, as a powder, or in particulate form. Particulate forms are preferred, including microparticles, which may be cross-linked or non-cross-linked and may be formed conveniently by spray-drying or lyophilization. See, e.g., He et al. (1999) Int. J Pharm. 187:53-65; He et al. (1999) J Microencapsul. 16:343-355. Other physical forms include gels, beads, films, sponges, fibres, emulsions, and the like.

Thus, the term “chitosan” as used with reference to the compositions, processes, methods and uses of the invention includes all forms and derivatives of chitosan.

C.3. Additional Adjuvants

The toxin and chitosan components act as mucosal adjuvants within the compositions of the invention. It is also possible to include one or more further mucosal adjuvants e.g.: (A) microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyortho ester, a polyanhydride, a polycaprolactone etc., such as poly(lactide-co-glycolide) etc.) optionally treated to have a negatively-charged surface (e.g. with SDS) or a positively-charged surface (e.g. with a cationic detergent, such as CTAB); (B) monophosphoryl lipid A mimics, such as aminoalkyl glucosaminide phosphate derivatives e.g. RC-529 (Johnson et al. (1999) Bioorg Med Chem Lett 9:2273-2278); (C) polyphosphazene (PCPP); (D) a polyoxyethylene ether or a polyoxyethylene ester (International patent application WO 99/52549); (E) a polyoxyethylene sorbitan ester surfactant in combination with an octoxynol (International patent application WO 01/21207) or a polyoxyethylene alkyl ether or ester surfactant in combination with at least one additional non-ionic surfactant such as an octoxynol (International patent application WO 01/21152); (F) an immunostimulatory oligonucleotide (e.g. a CpG oligonucleotide) and a saponin (International patent application WO 00/62800); and (G) liposomes (see, e.g., Chapters 13 & of Vaccine design: the subunit and adjuvant approach, eds. Powell & Newman, Plenum Press 1995 (ISBN 0-306-44867-X). Other mucosal adjuvants are also available (see, e.g. Chapter 7 of Vaccine design: the subunit and adjuvant approach, eds. Powell & Newman, Plenum Press 1995 (ISBN 0-306-44867-X)).

In addition to the mucosal adjuvants given above, the compositions of the invention may include one or more further adjuvants selected from the following group: (A) aluminum salts (alum), such as aluminum hydroxides (including oxyhydroxides), aluminum phosphates (including hydroxyphosphates), aluminum sulfate, etc (Chapters 8 & 9 of Vaccine design: the subunit and adjuvant approach, eds. Powell & Newman, Plenum Press 1995 (ISBN 0-306-44867-X)); (B) oil-in-water emulsion formulations (with or without other specific immunostimulating agents such as muramyl peptides [Muramyl peptides include N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.] or bacterial cell wall components), such as for example (a) MF59TM (Chapter 10 of Vaccine design: the subunit and adjuvant approach, eds. Powell & Newman, Plenum Press 1995 (ISBN 0-306-44867-X); International Publication WO 90/14837; U.S. Pat. No. 6,299,884) containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (C) saponin adjuvants (chapter 22 of Vaccine design: the subunit and adjuvant approach, eds. Powell & Newman, Plenum Press 1995 (ISBN 0-306-44867-X), such as QS21 or Stimulon™ (Cambridge Bioscience, Worcester, Mass.), either in simple form or in the form of particles generated therefrom such as ISCOMs (immunostimulating complexes; Chapter 23 of of Vaccine design: the subunit and adjuvant approach, eds. Powell & Newman, Plenum Press 1995 (ISBN 0-306-44867-X)), which ISCOMS may be devoid of additional detergent e.g. WO 00/07621; (D) Complete Freund's Adjuvant (CFA) and Incomplete Freund's Adjuvant (IFA); (E) cytokines, such as interleukins (e.g. IL-1, IL-2, IL-4, IL-5, IL-6, IL-7, IL-12, etc.) (International Publication W099/44636), interferons (e.g. gamma interferon), macrophage colony stimulating factor (M-CSF), tumor necrosis factor (TNF), etc.; (F) monophosphoryl lipid A (MPL) or 3-O-deacylated MPL (3dMPL) e.g. GB-2220221 and EP-A-0689454, optionally in the substantial absence of alum when used with pneumococcal saccharides e.g. International Publication WO 00/56358; (G) combinations of 3 dMPL with, for example, QS21 and/or oil-in-water emulsions e.g. EP-A-0835318; EP-A-0735898; and EP-A-0761231; (H) oligonucleotides comprising CpG motifs i.e. containing at least one CG dinucleotide, with 5-methylcytosine optionally being used in place of cytosine; (I) an immunostimulant and a particle of metal salt e.g. WO 00/23105; (J) a saponin and an oil-in-water emulsion e.g. WO 99/11241; (K) a saponin (e.g. QS21)+3dMPL+IL-12 (optionally+a sterol) e.g. WO 98/57659; (L) double-stranded RNA; (M) other substances that act as immunostimulating agents to enhance the effectiveness of the composition (e.g. chapter 7 of Vaccine design: the subunit and adjuvant approach, eds. Powell & Newman, Plenum Press 1995 (ISBN 0-306-44867-X)). With certain adjuvants, for example aluminum salts, the flu antigen(s) may be adsorbed to the aluminum salt.

D. Compositions and Preparations

The influenza antigen(s), detoxified ADP-ribosylating toxin and chitosan are typically prepared separately and then admixed to give a composition as described herein. The composition can then be presented and packaged in various ways.

Where compositions are for injection, they may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form for solution or suspension in liquid vehicles prior to injection.

Where the composition is for oral administration, for instance, it may be in the form of tablets or capsules (optionally enteric-coated), liquid, transgenic plants, drops, inhaler, aerosol, enteric coating, suppository, pessary, etc. (see, also Michetti (1998) J. Gastroenterol SuppIX:66-68 and Chapter 17 of Vaccine design: the subunit and adjuvant approach, eds. Powell & Newman, Plenum Press 1995 (ISBN 0-306-44867).

For mucosal routes, the composition is preferably adapted for and/or packaged for intranasal administration, such as by nasal spray, nasal drops, gel or powder. See, e.g., Almeida & Alpar (1996) J Drug Targeting 3:455-467; Agarwal & Mishra (1999) Indian J Exp Biol 37:6-16.

The compositions described herein can include various excipients, adjuvants, carriers, auxiliary substances, modulating agents, and the like. Preferably, the compositions will include an amount of the antigen sufficient to mount an immunological response. An appropriate effective amount can be determined by one of skill in the art.

As noted above, compositions of the invention can also contain liquids or excipients, such as water, saline, glycerol, dextrose, ethanol, or the like, singly or in combination, as well as substances such as wetting agents, emulsifying agents, or pH buffering agents.

Pharmaceutically acceptable carriers, which include any carrier that does not itself induce the production of antibodies harmful to the individual receiving the composition, may also be used in the compositions described herein. Suitable carriers are typically large, slowly metabolised macromolecules such as proteins, polysaccharides, polylactic acids, polyglycolic acids, polymeric amino acids, amino acid copolymers, trehalose (see, e.g., International Publication WO 00/56365), lipid aggregates (such as oil droplets or liposomes), and inactive virus particles. Such carriers are well known to those of ordinary skill in the art.

The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in Gennaro (2000) Remington.—The Science and Practice of Pharmacy. 20th ed ISBN: 0683306472.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art.

Compositions of the invention are preferably packaged in unit dose form. Effective doses can be routinely established. A typical human dose of the composition for intranasal use has a volume of between 0.1 and 0.5 ml e.g. two 100 μl sprays, one per nostril.

Within each dose, the amount of individual antigens can be varied and tested by routine methods. For intranasal administration, however, HA is typically between about 1 μg/dose and about 20 μg/dose (or any value therebetween), for example, 5 μg/dose, 7 μg/dose, 7.5 μg/dose, 10 μg/dose or 15 μg/dose. Compositions of the invention are preferably sterile. They are preferably pyrogen-free. They are preferably buffered e.g. at between pH 6 and pH 8, generally around pH 7. Where a composition comprises an aluminum hydroxide salt, it is preferred to use a histidine buffer. See, e.g., International Publication PCT/IB02/03495.

The compositions described herein can also be provided as kits, for example a kit comprising an influenza antigen, a detoxified ADP-ribosylating protein and a chitosan. One or more of the components may be freeze-dried and/or spray-dried for packaging into the kit. The components may be provided as a single composition or may be provided separately. Furthermore, the components may be reconstituted prior to use such that they are suitable for mucosal administration. The kits described herein may further include additional components such as syringes, reconstitution solutions, instruction manuals, and the like.

E. Administration

Compositions as described herein may be administered by various routes, including parenteral and mucosal administration. In certain embodiments, the compositions may be administered parenterally, for example by injection. Injection may be subcutaneous, intraperitoneal, intravenous or intramuscular. Intramuscular administration to the thigh is preferred. Needle-free injection may be used. A preferred route of mucosal administration is intranasal. Transdermal or transcutaneous administration is also possible (e.g. W0 98/20734).

In a preferred embodiment, the compositions are administered mucosally. Thus, the composition may thus be adapted for and/or packaged for mucosal administration. See, e.g., Walker (1994) Vaccine 12:387-400; Clements (1997) Nature Biotech. 15:622-623; McGhee et al. (1992) Vaccine 10:75-88. Methods of mucosal delivery are known in the art, for example as described in Remington's, supra. Of the various mucosal delivery options available, the intranasal route may be the most practical as it offers easy access with relatively simple devices that have already been mass-produced. Alternative routes for mucosal delivery of the composition are oral, intragastric, pulmonary, transdermal, intestinal, rectal, ocular, and vaginal routes. Furthermore, delivery of the compositions intranasally or orally may be preferred because influenza typically infects through nasal mucosa.

Combinations of various routes of mucosal administration and/or various routes of systemic administration can be used in order to induce optimal immunity and protection (both at the site the pathogen enters as well as at systemic sites where a mucosal pathogen has spread to. Additionally, mucosal administration eliminates the need for syringes or other administration devices.

When the compositions include one or more polynucleotides (e.g., sequences encoding one or more influenza antigens and/or one or more detoxified ADP-ribosylating proteins), any suitable means can be used for delivery including, but not limited to, use of liposomes, (Felgner et al. (1989) Proc. Natl. Acad. Sci. USA 84:7413-7417), microparticles (e.g., PLG described above), direct injection (Acsadi et al. (1991) Nature 352:815-818); gene and/or protein delivery vehicles and the like.

Administration may be a single dose schedule or a multiple dose schedule. A primary dose schedule may be followed by a booster dose schedule. Suitable timing between priming and boosting can be routinely determined. For example, a multiple dose schedule may be one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least in part, be determined by the potency of the modality, the vaccine delivery employed, the need of the subject and be dependent on the judgment of the practitioner.

Administration will generally be to an animal and, in particular, human subjects can be treated. The compositions are useful for vaccinating children and adults.

F. Medical Methods and Uses

Compositions described herein are useful in methods of generating an immune response (e.g., the compositions are immunogenic). The compositions may also be used as vaccines, e.g., as prophylactic (i.e. to prevent infection) or therapeutic (i.e. to treat disease after infection) compositions. Typically, the compositions are used prophylactically.

By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

Methods for determining immunogenicity of influenza vaccines are well known.

Thus, the invention provides a method of raising an immune response in a patient, comprising administering to a patient a composition of the invention. The immune response is preferably protective against influenza infection, and may comprise a humoral immune response and/or a cellular immune response.

The method may raise a booster response, in a patient that has already been primed against flu virus.

The invention also provides the use of (a) an influenza antigen, (b) a detoxified ADP-ribosylating toxin, and (c) chitosan, in the manufacture of a medicament for preventing influenza virus infection.

### EXAMPLES

### Example 1

### Intranasal Administration of Adjuvant Influenza Antigen

Nine groups of mice (10 mice to a group) were given two 10 μg intranasal doses at 4 week intervals of influenza virus hemagglutinin either alone, with a detoxified LT adjuvant, or with a detoxified LT adjuvant and 0.5% chitosan as shown in Table 1 below:

TABLE 1

| Group | Adjuvant |
|---|---|
| 1 | None |
| 2 | LTK63, 1 μg |
| 3 | LTK63, 10 μg |
| 4 | LTR72, 1 μg |
| 5 | LTR72, 10 μg |
| 6 | Chitosan + LTK63, 1 μg |
| 7 | Chitosan + LTK63, 10 μg |
| 8 | Chitosan + LTR72, 1 μg |
| 9 | Chitosan + LTR72, 10 μg |

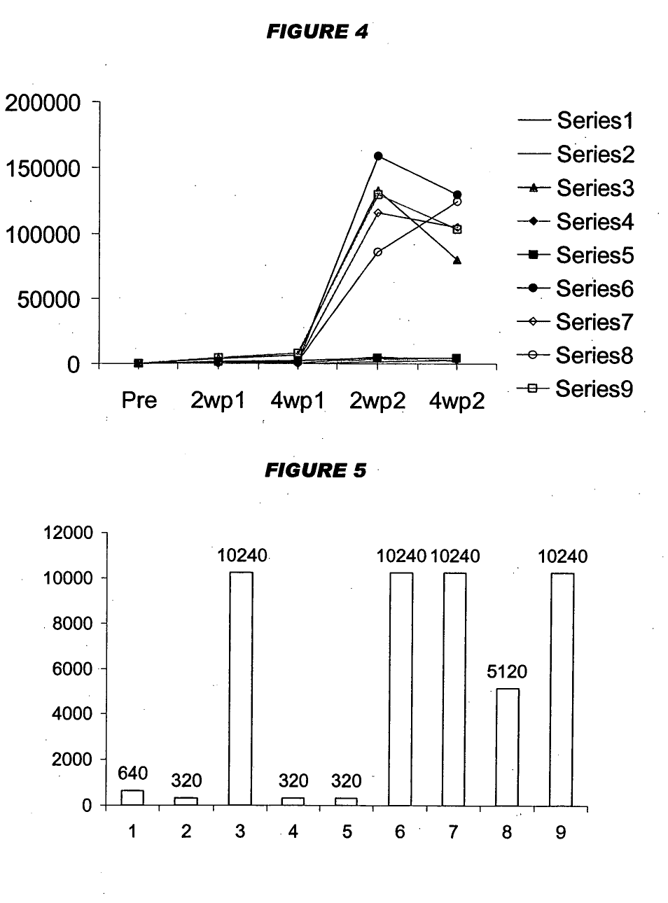

IgG titers were measured before immunization, and at 2 and 4 weeks after both doses. Results are shown in FIG. 4. Hemagglutination inhibition (HI) titers were measured 2 weeks after the second dose, and results are shown in FIG. 5.

A comparison of group 2 (HA+1 μg LTK63) and group 6 (HA+1 μg LTK63+0.5% chitosan) shows that the addition of chitosan gives substantially higher titers. The titers are increased to match those achieved with 10 μg LTK63. Thus, chitosan allows a lower dose of the detoxified ribosylating protein to be used.

It will be understood that the invention has been described by way of example only and modifications may be made whilst remaining within the scope and spirit of the invention.

1. A composition comprising an influenza antigen;
a detoxified ADP-ribosylating protein; and
a chitosan.

2. The composition of claim 1, wherein the influenza antigen comprises one or more surface antigens.

3. The composition of claim 2, wherein the surface antigens are haemagglutinin and/or neuraminidase.

4. The composition of claim 1, wherein the detoxified ADP-ribosylating protein is selected from the group consisting a detoxified diphtheria toxin protein, a detoxified exotoxin A protein, a detoxified cholera toxin (CT) protein; a detoxified heat-labile enterotoxin (LT) toxin protein; and a detoxified pertussis toxin (PT) protein.

5. The composition of claim 4, wherein the ADP-ribosylating protein is a detoxified LT, CT or PT protein.

6. The composition of claim 5, wherein the detoxified LT protein is selected from the group consisting of LTK7, LTX53, LTK63, LTY63, LTR72, LTX97, LTX104, LTS106, LTK112, LTG192, CTK7, CTK11, CTX53, CTF61, CTK63, CTY63, CTX97, CTX104, CTK112, CTS106, PTK9, PTG129 and combinations thereof.

7. The composition of claim 1, wherein the chitosan is at least 75% deacteylated.

8. The composition of claim 7, wherein the chitosan is alkylated.

9. The composition of claim 8, wherein the chitosan is trialkylated.

10. The composition of claim 1, wherein the influenza antigen is provided as a polynucleotide encoding the antigen.

11. The composition of claim 1, further comprising one or more additional antigens and/or one or more additional adjuvants.

12. The composition of claim 1, which is adapted for mucosal administration.

13. The composition of claim 12, wherein the mucosal administration is intranasal.

14. The composition of claim 13, in the form a nasal spray or nasal drops.

15. A dispensing device in combination of the composition of any of the preceding claims, wherein the dispensing device is adapted to deliver the composition intranasally.

16. A kit comprising the composition of claim 1, wherein when combined or reconstituted, the composition is suitable for mucosal administration.

17. A method of generating an immune response to influenza in a subject, the method comprising: administering to the subject the composition according to claim 1.

18. The method of claim 17, wherein the immune response protects the subject against influenza infection or disease.
